(19) United States
(12) Patent Application Publication
(10) Pub. No.: US 2006/0166968 A1
(43) Pub. Date: Jul. 27, 2006

(54) Crystalline form of oxcarbazepine

(76) Inventors: Sundaram Venkataraman, Hyderabad (IN); Nadimpally Satyavarahala Raju, Hyderabad (IN); Koilkonda Purandhar, Hyderabad (IN); Bairy Kondal Reddy, Medak (IN); Lekkala Amarnath Reddy, Hyderabad (IN); Golla China Mala Kondaiah, Hyderabad (IN)

Correspondence Address: DR. REDDY'S LABORATORIES, INC., 200 SOMERSET CORPORATE BLVD, SEVENTH FLOOR,, BRIDGEWATER, NJ, 08807-2862, US

(21) Appl. No.: 11/182,352

(22) Filed: Jul. 15, 2005

Related U.S. Application Data
(60) Provisional application No. 60/588,495, filed on Jul. 16, 2004.

(51) Int. Cl. A61K 31/55; C07D 223/18

(52) U.S. Cl. 514/217; 540/591

(57) ABSTRACT

Crystalline form F of oxcarbazepine.

### CROSS-REFERENCE TO RELATED APPLICATION

This application claims priority from copending U.S. Provisional Application No. 60/588,495 filed on Jul. 16, 2004, the entire content of which is hereby incorporated by this reference.

### INTRODUCTION TO THE INVENTION

The present invention relates to a crystalline form of oxcarbazepine. The present invention also relates to a process for the preparation of a crystalline form of oxcarbazepine.

Oxcarbazepine is chemically known as 10,11-Dihydro-10-oxo-5H-dibenz-[b,f]azepine-5-carboxamide, and its structural formula is:

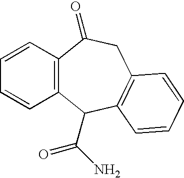

Oxcarbazepine is an antiepileptic drug available commercially as TRILEPTAL™. U.S. Pat. No. 3,642,775 claims oxcarbazepine specifically and discloses the process for the preparation of oxcarbazepine which comprises carbonylation of 10-methoxy-5H-dibenz[b,f]azepine with phosgene gas, giving 10-methoxy-5H-dibenz[b,f]azepine-5-carbonyl chloride, and ammonolysis of the resultant compound to give the amide followed by hydrolysis to give oxcarbazepine.

U.S. Patent Application No. 2003/0004154 A1 discloses the new crystal forms B, C, and D, and also the process for the preparation of polymorphs form B, form C and form D.

The process for the preparation of form B comprises preparing a solution of oxcarbazepine in dichloromethane and adding the solution to toluene followed by stirring for 5 minutes, finally evaporating the solvent at the rate of 5 g/minute;

Alternatively, the form B of oxcarbazepine can be prepared by dissolving the oxcarbazepine in toluene at room temperature followed by refluxing for five minutes, then cooling the mixture immediately to 0° C., and finally filtering after 5 minutes under reduced pressure.

The process for the preparation of form C comprises dissolving the oxcarbazepine in toluene at room temperature, refluxing for 10 minutes, cooling to 0° C. at the rate of 40° C. per minute, and finally filtering under reduced pressure.

The process for the preparation of form D comprises dissolving the oxcarbazepine in toluene at room temperature, refluxing for 5 minutes, cooling to 0° C. and finally evaporating the solvent.

U.S. Patent Application No. 2003/0004154 A1 also discloses the solvated form E of oxcarbazepine and a process for its preparation. The process for the preparation of solvated form E comprises dissolving oxcarbazepine in chloroform at room temperature, heating to about 55° C. for 5 minutes, cooling to 21.5° C., and after 8 hours cooling to 16° C. After 48 hours the suspension was heated to 25° C. and finally filtered under reduced pressure.

Further, U.S. Patent Application No. 2003/0004154 A1 also discloses that the crystal forms B, C, and D, and solvated form E of oxcarbazepine, can be converted into form A under various conditions.

Polymorphism is the occurrence of different crystalline forms of a single compound and it is a property of some compounds and complexes. Thus, polymorphs are distinct solids sharing the same molecular formula, yet each polymorph can have distinct physical properties. Therefore a single compound may give rise to a variety of polymorphic forms where each form has different and distinct physical properties, such as different solubility profiles, different melting point temperatures, and/or different X-ray diffraction patterns. Since the solubility of each polymorph may vary, identifying the existence of pharmaceutical polymorphs is essential for providing pharmaceuticals with predictable solubility profiles. It is desirable to investigate all solid forms of a drug, including all polymorphic forms, and to determine the stability, dissolution and flow properties of each polymorphic form. Polymorphic forms of a compound can be distinguished in the laboratory by X-ray diffraction techniques and by other methods, such as infrared spectrometry.

However, it is important to further evaluate polymorphism for a compound to obtain new polymorphs exhibiting different dissolution characteristics and in some cases superior bioavailability, stability and excellent handling properties.

Hence, one aspect of the present invention is to provide a new crystalline form of oxcarbazepine. The new crystalline form of oxcarbazepine of the present invention will hereinafter sometimes be designated as “form F of oxcarbazepine,” for convenience.

Another aspect of the present invention is to provide a process for the preparation of novel crystalline form F of oxcarbazepine.

The process for the preparation of crystalline form F of oxcarbazepine of the present invention is simple, non-hazardous, and commercially viable.

### SUMMARY OF THE INVENTION

In one aspect, the invention includes crystalline form F of oxcarbazepine.

In another aspect, the invention includes a process for preparing crystalline form F of oxcarbazepine, comprising providing a solution of oxcarbazepine in an organic solvent, cooling the solution, and recovering crystalline form F of oxcarbazepine.

### BRIEF DESCRIPTION OF THE DRAWINGS

FIG. 1 is an X-ray powder diffraction pattern of the crystalline form F of oxcarbazepine prepared according to Example 1.

FIG. 2 is an Infrared absorption spectrum in potassium bromide of the crystalline form F of oxcarbazepine prepared according to Example 1.

FIG. 3 is a differential scanning calorimetric analysis of crystalline form F of oxcarbazepine prepared according to Example 1.

### DETAILED DESCRIPTION

The present invention comprises crystalline form F of oxcarbazepine and a process for the preparation thereof.

Accordingly, an embodiment of a process for the preparation of form F of oxcarbazepine comprises dissolving oxcarbazepine in an organic solvent, cooling the solution to produce a solid, and recovering the solid product.

Any form of oxcarbazepine is acceptable for use as a starting material in the process of this invention. This includes, without limitation, any amorphous or crystalline forms, or any solvates, hydrates, or anhydrous forms.

Organic solvents that are useful for preparing form F of oxcarbazepine are generally any solvents in which oxcarbazepine is soluble. Examples of such solvents are, but are not limited to: alcoholic solvents such as methanol, ethanol, propanol, butanol, and the like; halogenated solvents such as dichloromethane, chloroform, ethylene dichloride, and the like; aliphatic solvents such as n-butane, n-pentane, n-hexane, n-heptane and the like; ketonic solvents such as acetone, ethyl methyl ketone, and the like; esters such as ethyl acetate, n-butyl acetate, t-butyl acetate, and the like; ether solvents such as diethyl ether, di-isopropyl ether, and methyl tertiary butyl ether; tetrahydrofuran; and hydrocarbon solvents such as toluene, xylene, and the like. Mixtures of any two or more solvents from a chemical class, as well as mixtures of solvents from different chemical classes, are useful in the invention.

In one embodiment the solution of oxcarbazepine can be prepared by dissolving the oxcarbazepine in toluene at elevated temperatures ranging from about 80 to 160° C., or from about 120 to 145° C., or from about 140 to 145° C., in an autoclave under elevated pressure ranging from 0.5 to 5 Kg/cm², or 1 to 3 Kg/cm², such as over a period of about 0.5 to 4 hours, or about 1 to 2 hours.

By using elevated temperatures and pressures for forming the solution, a higher concentration of solute will be obtained, and this improves the yield of the desired product. An alternative for accomplishing this result is to prepare a solution and then concentrate it by solvent removal, such as by heating under a vacuum to reduce the time required for this operation.

The resulting solution can optionally be filtered to remove any undissolved particles, using various filtration techniques such as pressure filtration, gravity filtration, vacuum filtration, and other techniques that are familiar to those skilled in the art. In addition, techniques such as centrifugation are useful for separating the solid from the liquid. This separation will preferably be conducted under temperature and other conditions as were used for the preparation of the solution.

The reduced temperatures that are used to produce form F crystals of oxcarbazepine can be varied from about −10 to 40° C., or about 0 to 10° C., or about 0 to 5° C. Those skilled in the art will be able to determine an optimal time for crystallization to obtain a desired product recovery, by simple experimentation.

After the crystals form, product is recovered by filtration, centrifugation, etc., as described above for solution clarification.

The recovered solid can optionally be dried by using conventional methods known in the art, such as drying by applying vacuum, suction drying, air drying, oven drying, fluid bed drying, spin flash drying, or other techniques. The solid can be dried at temperatures from about 20 to 70° C., or 40 to 45° C. or at room temperatures.

The X-ray powder diffraction pattern of the form F of oxcarbazepine was determined using a Bruker Axe, DS Advance Powder X-ray powder diffractometer with a Cu K alpha-1 radiation source. The characteristic peaks (in 2θ degrees) and their relative intensities (in percentages) are given in the following Table 1.

TABLE 1

| Diffraction angle (2θ°) | Intensity (I/I₀), % |
|---|---|
| 8.9 | 36.4 |
| 11.2 | 57.8 |
| 11.9 | 41.8 |
| 13.7 | 15.4 |
| 15.9 | 100 |
| 16.8 | 26.5 |
| 17.7 | 25.4 |
| 18.6 | 6.3 |
| 20.2 | 14.3 |
| 20.6 | 10 |
| 22.5 | 38 |
| 24.4 | 13.5 |
| 24.6 | 18.7 |
| 25.2 | 13.7 |
| 26.1 | 14.9 |
| 26.5 | 28.5 |
| 26.9 | 11.7 |
| 27.7 | 13.4 |
| 30.1 | 6.8 |
| 34.1 | 8.2 |

Some variation in the location of peaks is typically observed in any instrumental analytical method, resulting from factors such as tolerances in the instrument construction, sample preparation differences, and operator technique. For X-ray diffraction results, the locations of peaks for a substance can vary by ±0.1° 2θ or more, particularly when samples have been analyzed on more than one instrument. However, the similarities between patterns will be apparent to a person skilled in the art, for a given substance. Peak heights are subject to considerable variation, as these are more dependent on sample preparation techniques, so the relative locations of the peaks is a more important parameter than their heights.

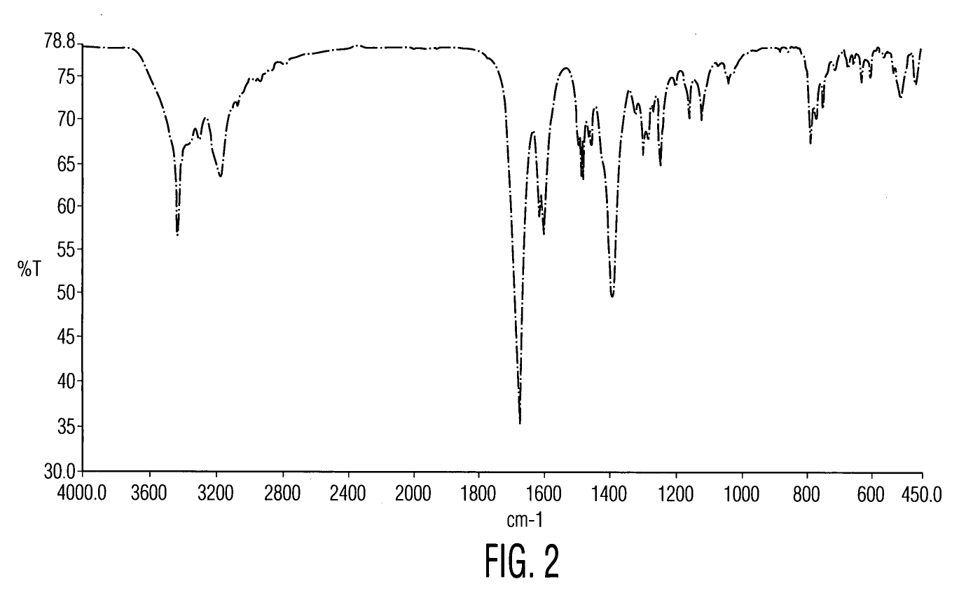

Crystalline form F of oxcarbazepine of the present invention has also been characterized by infrared spectrophotometry, using the KBr pellet transmission method. The Infrared spectrum of the crystalline form F of oxcarbazepine is as depicted in FIG. 2. The identified significant infrared absorption peaks are at about 3427, 3297, 3167, 1674, 1611, 1597, 1489, 1450, 1476, 1388, 1315, 1276, 1288, 1236, 1191, 1149, 1112, 1028, 781, 762, 742, 707, 668, 600, 627, 651, 466, and 511, ±2 cm⁻¹.

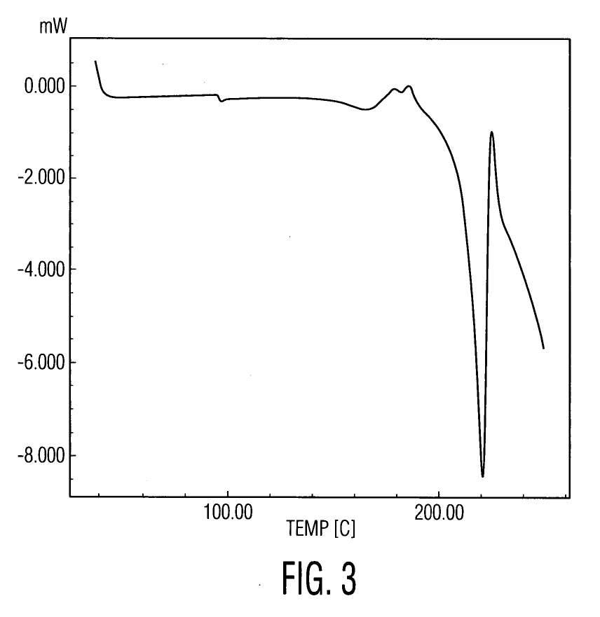

The crystalline form F of oxcarbazepine has further been characterized by differential scanning calorimetry, and exhibits a significant endothermic peak about 217° C. The differential scanning calorimetry thermogram of crystalline oxcarbazepine form F is substantially as depicted in FIG. 3.

The process for preparing crystalline form F of oxcarbazepine of the present invention is cost effective, non hazardous and easily scalable. Form F of oxycarbazepine is useful for preparing pharmaceutical compositions such as tablets, capsules, syrups, elixirs, and injectable solutions. Tablets and other solid compositions frequently contain at least one from the classes binders, diluents, glidants, lubricants, and other excipients. Liquid compositions frequently contain at least one excipient from classes such as suspending agents, sweeteners, taste masking agents, preservatives, and others. The choice of particular excipients is well known to those having skill in the art.

The invention will be explained in more detail with reference to the following examples, which are provided by way of illustration only and should not be construed as limiting the scope of the invention in any manner.

### EXAMPLE 1

Crystalline Form-F of Oxcarbazepine

7.8 g of oxcarbazepine and 1 liter of toluene were placed in an autoclave vessel and heated to a temperature of about 140-145° C. under a reduced pressure of about 2 to 3 Kg/cm² for complete dissolution. The reaction solution was than stirred for about 30 minutes followed by filtering the reaction mass at the same temperature by a pressure filter. The filtrate was cooled to a temperature of about 2° C. with simultaneous stirring at the same temperature for about 60 minutes. The formed solid was filtered and washed with 20 ml of toluene followed by suction drying. The solid mass was further subjected to drying at a temperature of about 58 to 65° C. under a vacuum of about 600 to 750 mm Hg to obtain the desired crystalline form F of oxcarbazepine.

### EXAMPLE 2

Crystalline Form-F of Oxcarbazepine

7.8 g of oxcarbazepine and 1 liter of toluene were placed in an autoclave vessel and heated to a temperature of about 140-145° C. under a reduced pressure of about 2 to 3 Kg/cm² for complete dissolution. The reaction solution was than stirred for about 30 minutes followed by filtering the reaction mass at the same temperature using a pressure filter. The filtrate was cooled to temperature of about 2° C. with simultaneous stirring at the same temperature for about 60 minutes. The formed solid was filtered and washed with 20 ml of toluene followed by suction drying at a temperature of about 27° C. to obtain the desired crystalline form F of oxcarbazepine.

We claim:

1. Crystalline form F of oxcarbazepine.

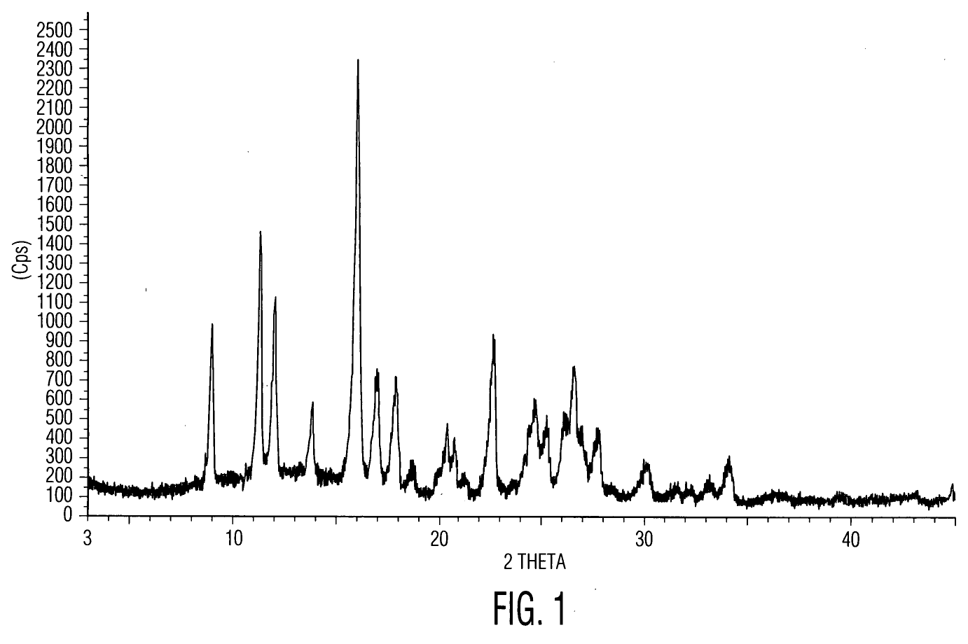

2. The crystalline form F of oxcarbazepine of claim 1, having an X-ray diffraction pattern substantially in accordance with FIG. 1.

3. The crystalline form F of oxcarbazepine of claim 1, having an X-ray powder diffraction pattern using Cu K alpha 1 radiation with peaks about 8.9, 11.2, 11.9, 13.7, 15.9, 16.8, 17.7, 18.6, 20.2, 20.6, 22.5, 24.4, 24.6, 25.2, 26.1, 26.5, 26.9, 27.7, 30.1, 30.1, 34.1±0.1 degrees 2θ.

4. The crystalline form F of oxcarbazepine of claim 1, having an infrared absorption spectrum substantially in accordance with FIG. 2.

5. The crystalline form F of oxcarbazepine of claim 1, having infrared absorption peaks about 3427, 3296, 3167, 1674, 1611, 1597, 1489, 1450, 1476, 1388, 1315, 1276, 1288.3, 1236, 1191, 1149, 1112, 1028, 781, 762, 742, 707, 668, 600, 627, 651, 466, and 511, ±2 cm⁻¹.

6. The crystalline form F of oxcarbazepine of claim 1, having a differential scanning calorimetry thermogram substantially in accordance with FIG. 3.

7. The crystalline form F of oxcarbazepine of claim 1, having a differential scanning calorimetry thermogram exhibiting an endothermic peak about 217° C.

8. A process for preparing a crystalline form F of oxcarbazepine, comprising providing a solution of oxcarbazepine in an organic solvent, cooling the solution, and recovering crystalline form F of oxcarbazepine.

9. The process of claim 8, wherein the organic solvent comprises a hydrocarbon, an ester, an alcohol, a halogenated hydrocarbon, an ether, or a mixture of any two or more thereof.

10. The process of claim 8, wherein the organic solvent comprises toluene, xylene, or a mixture thereof.

11. The process of claim 8, wherein the organic solvent comprises toluene.

12. The process of claim 8, wherein the solution is cooled to about −10 to 40° C.

13. The process of claim 8, wherein the solution is cooled to about 0 to 10° C.

14. The process of claim 8, wherein the solution is cooled to about 0 to 5° C.

15. Crystalline form F of oxcarbazepine, prepared by the process of claim 8.

16. The process of claim 8, wherein an organic solvent comprises toluene and the solution is cooled to about 0 to 5° C.

17. The process of claim 8, wherein oxcarbazepine is dissolved at an elevated temperature and an elevated pressure.

18. The process of claim 8, wherein oxcarbazepine is dissolved in an organic solvent comprising toluene at an elevated temperature and an elevated pressure, and the solution is cooled to about 0 to 5° C.

19. A pharmaceutical composition, comprising crystalline form F of oxcarbazepine prepared by the process of claim 8, and at least one pharmaceutical excipient.

20. A pharmaceutical composition, comprising crystalline form F of oxcarbazepine and at least one pharmaceutical excipient.
